(19) United States
(12) Patent Application Publication
(10) Pub. No.: US 2014/0088370 A1
(43) Pub. Date: Mar. 27, 2014

(54) ADJUSTABLE SURGICAL SUPPORT AND RETRACTOR SYSTEM

(71) Applicant: AUTOMATED MEDICAL PRODUCTS CORPORATION, Sewaren, NJ (US)

(72) Inventors: PIER CRISTOFORO GIULIANOTTI, Chicago, IL (US); Jerry M. Brown, Sewaren, NJ (US); Adrian Greda, Harwood Heights, IL (US); Krzysztof S. Kubala, Wheeling, IL (US); Roberta Higham, Wayne, IL (US)

(73) Assignee: AUTOMATED MEDICAL PRODUCTS CORPORATION, Sewaren, NJ (US)

(21) Appl. No.: 14/027,889

(22) Filed: Sep. 16, 2013

Related U.S. Application Data
(62) Division of application No. 12/895,850, filed on Sep. 30, 2010, now abandoned.
(60) Provisional application No. 61/329,537, filed on Apr. 29, 2010.

(51) Int. Cl. A61B 17/02; A61B 1/32; A61B 19/00

(52) U.S. Cl. CPC A61B 17/0206; A61B 19/26; A61B 1/32
USPC 600/228

(57) ABSTRACT

A surgical support system including a primary telescoping support; and a secondary telescoping support that is telescopically mounted to the primary support and is configured for telescopically mounting a surgical tool thereto, such that such that telescoping of the secondary support with respect to the primary support repositions the secondary telescoping support. The secondary support is mounted to the primary support telescopably along a first telescoping axis and is configured for mounting the surgical tool telescopably along a second telescoping axis that is oriented at a non-parallel angle to the first telescoping axis. The secondary support includes a first mounting portion; a second mounting portion configured for telescopically mounting the surgical tool; and an articulable joint disposed for adjusting the angle between the first and second telescoping axes. The articulable joint is releasably lockable for locking the angle. The secondary support comprises a flared end for facilitating reception of the surgical tool.

### CROSS-REFERENCE TO RELATED APPLICATIONS

The present application is a divisional of U.S. patent application Ser. No. 12/895,850, filed on Sep. 30, 2010, which claims the benefit of priority to U.S. patent application Ser. No. 61/329,537, filed on Apr. 29, 2010, each of which are herein incorporated by reference in their entirety. Further, the present application also hereby incorporates by reference the entire contents of U.S. patent application Ser. No. 11/166,170 and U.S. patent application Ser. No. 11/165,064 and U.S. Pat. Nos. 4,796,846 and 6,315,260.

### TECHNICAL FIELD

The present disclosure relates generally to an adjustable system for supporting surgical tools.

### BACKGROUND

Surgical tool support systems are used to securely and stably maintain surgical tools, such as surgical retractors, in a fixed position so that a physician or physician's assistant does not have to manually hold the tools throughout the duration of the surgical procedure. Some known surgical tool support systems include adjustable components which are removably mounted or other wise attached to a surgical tool support or a horizontal bar, which is positioned over the patient and further connects in a fixed manner to the operating room table. An example of a surgical tool support is described in U.S. Pat. No. 4,143,652, the content of which is hereby incorporated herein by reference thereto.

Surgical tools that are commonly held by such surgical tool support systems include, for example, retractors for retracting internal organs, and other structures, such as a patient's ribcage. The Stieber Rib Grip Kit™ sold by Automated Medical Products Corp. is used, for instance, for retracting the ribs during abdominal surgery to retract the upper middle abdomen of a patient.

U.S. Pat. No. 6,302,843 described a tool holder platform that can be mounted to horizontal bar that is positioned above a patient to hold a hydra, which, with various support arms, supports other tools such as a retractor to retract and maintain organs in the retracted position.

### SUMMARY

The present disclosure relates generally to an adjustable system for supporting surgical tools. A surgical support system including a primary telescoping support; and a secondary telescoping support that is telescopically mounted to the primary support, the secondary support being configured for telescopically mounting a surgical tool thereto, such that such that telescoping of the secondary support with respect to the primary support repositions the secondary telescoping support. The secondary support is mounted to the primary support telescopably along a first telescoping axis; and the secondary telescoping support is configured for mounting the surgical tool telescopably along a second telescoping axis that is oriented at a non-parallel angle to the first telescoping axis. The secondary support includes a first mounting portion; a second mounting portion configured for telescopically mounting the surgical tool; and an articulable joint disposed between and associating the first mounting portion and the second mounting portion for adjusting the angle between the first and second telescoping axes. The articulable joint is releasably lockable for locking the angle. The secondary support comprises a flared end for facilitating reception of the surgical tool into the telescopic mounting with the secondary support.

The system may further include a retractor telescopically mounted to the secondary support. Each of the primary supports comprises an engagement member configured for selectively preventing telescopic extension of the second support and surgical tool, respectively. At least one of the engagement members has a configuration in which it allows telescopic retraction of the secondary support or surgical tool, respectively, while preventing the telescopic extension thereof. The secondary support comprises a rack that includes teeth, and the primary support include the at least one engagement member, which engagement member comprises a ratchet engageable with the teeth to selectively prevent telescopic extension thereof while allowing retraction thereof. Each engagement member has a configuration in which it allows telescopic retraction of the second support and surgical tool, respectively, while preventing the telescopic extension thereof. The secondary support comprises a rack that includes teeth, and the primary support includes a ratchet member engageable with the teeth to selectively prevent telescopic extension thereof while allowing retraction thereof; and the secondary support member comprises a ratchet member configured for engagement with teeth of a rack of the surgical tool to selectively prevent telescopic extension thereof while allowing retraction thereof.

The system may further include a handle configured for telescopically mounting the surgical tool thereto and including an engagement member for selectively preventing telescopic extension of the surgical tool rack. The secondary support comprises a rack including teeth, and the primary support includes the engagement member, which comprises a pinion for telescopically drawing or extending the rack. The system may further include a base member to which the primary support is mountable and which is configured for securing to a surgical table. The base member comprises a rail clamp that is configured for clamping to a surgical-table rail. The primary support comprises a support rod configured for securing to the rail clamp and positioning the secondary surgical support over the table.

Further disclosed herein is a method of conducting surgery, including retracting tissue with the retractor of the surgical support system of claim 6; and conducting robotic surgery wile the tissue is retracted.

Further disclosed herein is a retractor, including a substantially straight retractor blade having a length of at least about 20 centimeters and configured for retracting tissue; a supporting portion extending in a first direction for supporting and pulling the retractor blade; and an elbow having a radius of less than about 7 millimeters that connects the mounting portion to the blade at an angle to each other. The blade is substantially flat. The retractor may further include an end portion extending at an angle from the blade and including a surgical instrument mounted to the end portion and configured for performing an operation. The supporting portion comprises a rack for telescopic mounting to a surgical support system. The supporting portion may include a holding portion extending away from an abutting surface of the blade, which is configured for abutting and retracting tissue; and a diagonal portion disposed between the elbow and the blade at an acute angle with respect to the blade to over at least about 20% of the blade length from the elbow.

Further disclosed herein is a retractor, including a retractor blade having an abutting surface configured for abutting and retracting tissue; a surgical instrument disposed on the blade configured for performing an operation; and an instrument connector extending along the abutting surface to the surgical instruments. It may also include a lip extending at an angle from a tip of the blade, wherein the surgical instruments are mounted on the lip.

Further described herein is a surgical support, comprising a telescoping support defining a telescopic bore configured for telescopically receiving a surgical tool, the support including a flared inlet guide leading to the telescopic bore for facilitating reception of a surgical tool into telescopic association with the support.

Further described herein is a retractor holder, including a handle shaft configured for grasping with a hand, the handle shaft defining an internal telescopic bore configured for receiving and guiding a surgical retractor along a telescopic axis; and a ratchet member associated with the handle shaft for racheting engagement with teeth of a mounting portion of the surgical retractor to selectively prevent telescopic extension thereof while allowing telescopic retraction.

While multiple embodiments are disclosed, still other embodiments of the present disclosure will become apparent to those skilled in the art from the following detailed description, which shows and describes illustrative embodiments. As will be realized, the invention is capable of modification in various aspects, all without departing from the spirit and scope of the present disclosure. Accordingly, the drawings and detailed descriptions are to be regarded as illustrated in nature, and not restrictive.

### BRIEF DESCRIPTION OF THE FIGURES

While the specification concludes with claims particularly pointing out and distinctly claiming the subject matter that is regarded as forming the various embodiments of the present disclosure, it is believed that the embodiments will be better understood from the following description taken in conjunction with the accompanying Figures, in which:

FIG. 1 depicts one embodiment of an adjustable surgical support system in accordance with the present disclosure;

FIG. 2 depicts another embodiment of an adjustable surgical support system in accordance with the present disclosure;

FIG. 3 depicts an example horizontal support component of the surgical support system shown in FIG. 1;

FIG. 4 depicts the interior of a horizontal bar guide component thereof;

FIG. 5 depicts an example surgical retractor support component the surgical support system shown in FIG. 1;

FIG. 6 depicts an example surgical retractor support component the surgical support system shown in FIG. 2;

FIG. 7 depicts an example surgical retractor suitable for use with an adjustable surgical support system in accordance with the present disclosure;

FIG. 8 depicts another of the surgical retractor shown in FIG. 7; and

FIGS. 9a-c are diagrams of the surgical retractor shown in FIG. 1.

FIG. 10 depicts an embodiment of a retractor handle suitable for use with the adjustable surgical support system in accordance with the present disclosure.

FIG. 11 depicts an axial view of the retractor handle shown in FIG. 10.

FIG. 12 depicts a retractor handle connected to a retractor in a cross-section view.

FIG. 13 depicts an example retractor having an angled side.

### DETAILED DESCRIPTION

While multiple embodiments are disclosed, still other embodiments of the present disclosure will become apparent to those skilled in the art from the following detailed description, which shows and described illustrative embodiments. As will be realized, the invention is capable of modification in various aspects, all without departing from the spirit and scope of the present disclosure. Accordingly, the drawings and detailed descriptions are to be regarded as illustrated in nature, and not restrictive.

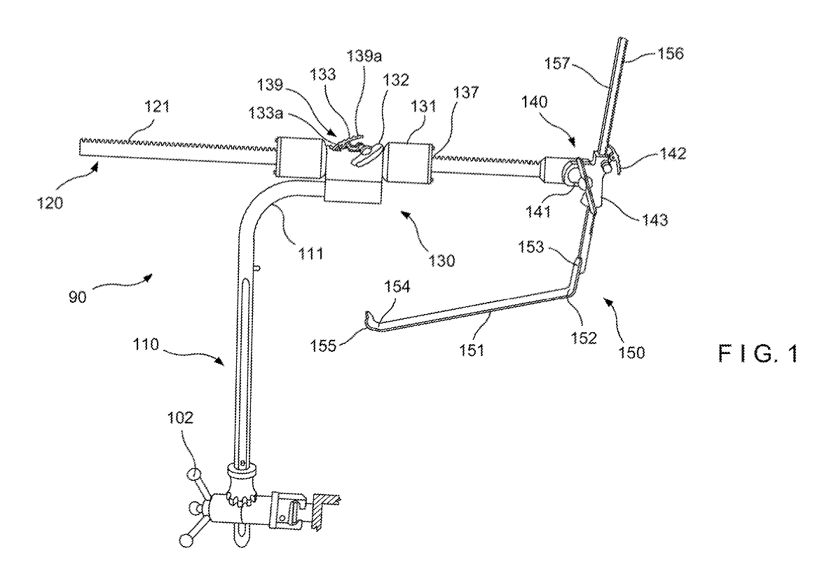

The present disclosure relates generally to an adjustable surgical support system and retractors that can be used therewith or independently. Referring to the one embodiment depicted in FIG. 1, an adjustable surgical retractor system 90 is shown mounted on an operating room table 100 having a rail 101. A mounting member, such as a rail clamp or swinger clamp 102, is adjustable attached to the operating table rail 101. Suitable rail clamps 102 are described in U.S. Application Publication No. 2006/0290076, and U.S. Pat. Nos. 4,796,846 and 6,315,260, the contents of which are herein incorporated by reference in their entirety.

A surgical tool support 110 is depicted adjustably positioned within the clamp 102. The surgical tool support 110, depicted in the embodiment of FIG. 1, is preferably of an L-shape, with a bend 111 transitioning fixedly at approximately 90° at the upper end of the support. Other suitable surgical tool supports, including adjustable surgical tool supports, are described in U.S. Application Publication 2006/0293568, the contents of which are herein incorporated by reference in their entirety. In a preferred embodiment, the length of the surgical tool support 110 may be 665 mm. In other embodiments, the length may be between 600 and 700 mm, or between 500 and 800 mm. Alternative supports can have other shapes, and can be straight or can include an attachment to a rail or another surgical support member.

At the top end of the surgical tool support 110 is depicted a first telescoping support component, which can be a horizontal support component 130 placed so as to extend over and across the patient on table 100. The preferred embodiment of the horizontal support component 130 includes a horizontal bar guide and a horizontal bar 120 to extend over the patient. The horizontal bar 120 includes a plurality of teeth 121, such as on the upper surface thereof. The horizontal bar 120 is inserted through the horizontal bar guide 131. The horizontal bar guide 131 includes an opening shaft 137 through which the horizontal bar 120 is received. Opening 137 can be a cylindrical bore or can have another shape, depending on the cross-sectional shape of bar 120.

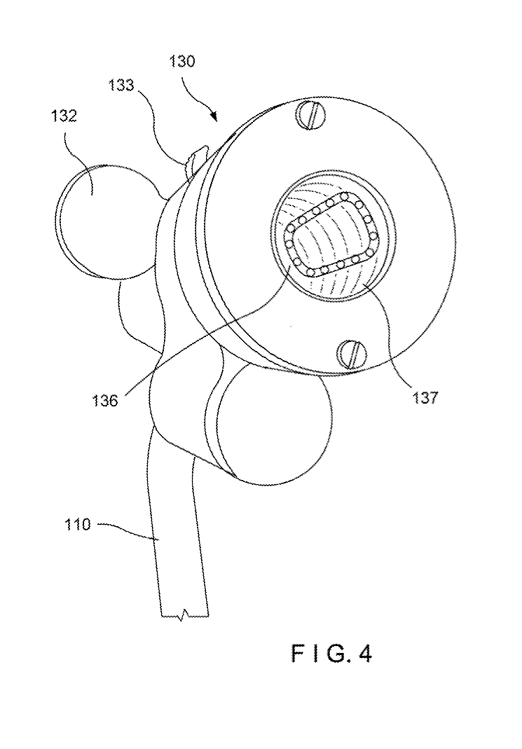

As shown in FIG. 4, the interior of the opening 137 of the horizontal support component 130 can have bearings to facilitate sliding of the bar 120. Ball bearings are shown in elliptical races 136, although other types of bearings, including bushings can be used, and in one embodiment, a simple metal bore is provided.

The teeth 121 of the horizontal bar 120 cooperates with an adjustment component 132 of the horizontal bar guide. The adjustment component 132, in one embodiment, can include a pinion to engage the teeth 121 of the horizontal bar 121 to provide a rack and pinon to move, such as to telescope, the bar 120 for adjusting the position of the horizontal bar 120 horizontally. A manipuable portion 132 of the horizontal support component 130 is disposed and configured to be exterior of the horizontal bar guide 131, connected with the pinion which extends into the interior of the bar guide 131 to operate the rack and pinion. Alternative embodiments can have other mechanisms to move, position, and/or lock the bar 120 with respect to the support 110, such as with a pure ratchet or other arrangements.

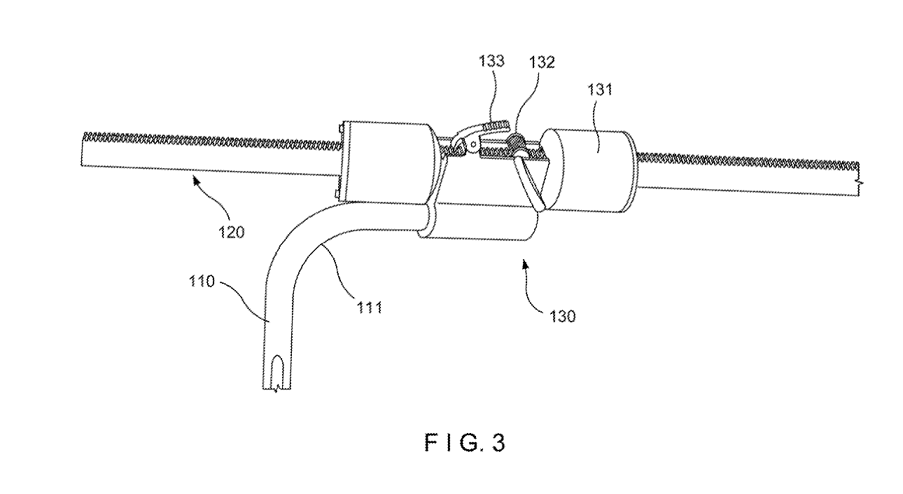

FIG. 3 depicts a close-up view of the horizontal support component 130 of the embodiment of FIG. 1. The teeth 121 of the horizontal bar 120 are showing greater detail, as is the cylindrical shape of the horizontal bar guide 131. The ratchet 133 is also visible with the teeth contact and being within the support component 130, and the finger pad 139a being exterior. The adjustment component 132, the interior portion of which in this embodiment is a pinion for cooperation with the rack (teeth 121) of the horizontal bar 120, is also shown on the horizontal support component 130.

Further included on the horizontal support component 130 is a ratchet 139 mechanism. The preferred ratchet includes a pivoting jaw 133 that is spring-loaded or otherwise biased into racheting engagement with the teeth 121 of the horizontal bar 120. The contact point between the jaw 133 and the teeth 121 may be in the form of a pawl. The teeth 121 and the ratchet jaw 133 cooperate in such a manner that when the ratchet is in contact with the teeth, the horizontal bar 120 can only be moved in one direction, preferably distally with respect to the incision, towards the upright support 110. Movement in the other direction, proximally with respect to the incision, is prevented by the ratchet when it is engaged. In this manner, tension provided by tissue that is being held by a retractor on the far end of the bar 120 is resisted, but adjustment of the bar in the other direction is quick and easy without disengaging the ratchet. To move the horizontal bar 120 in the distal direction (proximally, away from the rail clamp), the ratchet is lifted by a user out of engagement with the teeth 121, such as by depressing on a finger pad 139a on an opposite side of the ratchet pivot 133 a from the jaw 133, thereby allowing the horizontal bar 120 to be moved in either direction. The rack and pinion, or other incremental adjustment mechanism to move the bar 120, is preferably configured to allow the bar 120 to be moved by directly pulling on the bar distally, towards the rail clamp. In a preferred embodiment the length of the horizontal bar 120 may be 440 mm. In other embodiments, the length of the horizontal bar 120 may be between 400 mm and 480 mm, or between 350 mm and 550 mm.

At one end of the horizontal bar 120 is a secondary adjustment member 140. In some embodiments, the secondary adjustment member 140 may include mechanisms similar to the horizontal support component 130. The secondary adjustment member 140 may include an opening therethrough for accepting an mounting portion 157 of a surgical retractor 150. The mounting portion 157 of the surgical retractor 150 may have teeth 156 on an outward surface thereof similar to teeth 121. An adjustment component 141 of the secondary adjustment member 140 may be configured similar to the adjustment component 132 of the horizontal support component 130. Some embodiments can include, for example, a rack and pinion and/or a ratchet relationship between the adjustment component 141 and the teeth 156, or any other known adjustable relationship or configuration. The preferred embodiment is shown with a ratchet 142 of the secondary adjustment member 140 may cooperate with the teeth 156 of the mounting portion 157 of the retractor 150 a similar manner as with the ratchet 133 and the horizontal bar 120. The secondary adjustment member may also be rotatably adjustable in the plane of the horizontal bar about its connection point to the horizontal bar 120. The degree of rotatable adjustability may be between +/−20°, +/−40°, or +/−70° from vertical. The length of the mounting portion 157 of the retractor 150 may preferably be 150, 170, or 200 mm. In some embodiments, the length may be between 130 and 220 mm, or it may be between 100 and 250 mm.

In preferred embodiments, an insertion end 143 of the secondary adjustment member is flared so as to easily receive the mounting portion 157 of the retractor 150. While the axial cross-section of the opening of the secondary adjustment member is preferably shaped to prevent or limit axial rotation of the retractor, the flared end can flare from that shape to another to easily receive the tip of the mounting portion 157. For instance, in the embodiment shown, the mounting portion 157 cross-section is generally square, as is the cross-section of the mounting portion 157, and the flared guide end becomes generally rounded or circular with a substantially larger cross section, e.g. about 2 to 10 times as large as the cross section holding the adjustment member from rotations. During surgery, a surgical team will position the retractor within the patient, and then may easily position and adjust the surgical retractor system so that the insertion end 143 of the vertical support component is placed over the mounting portion 157 to receive and secure the retractor 150.

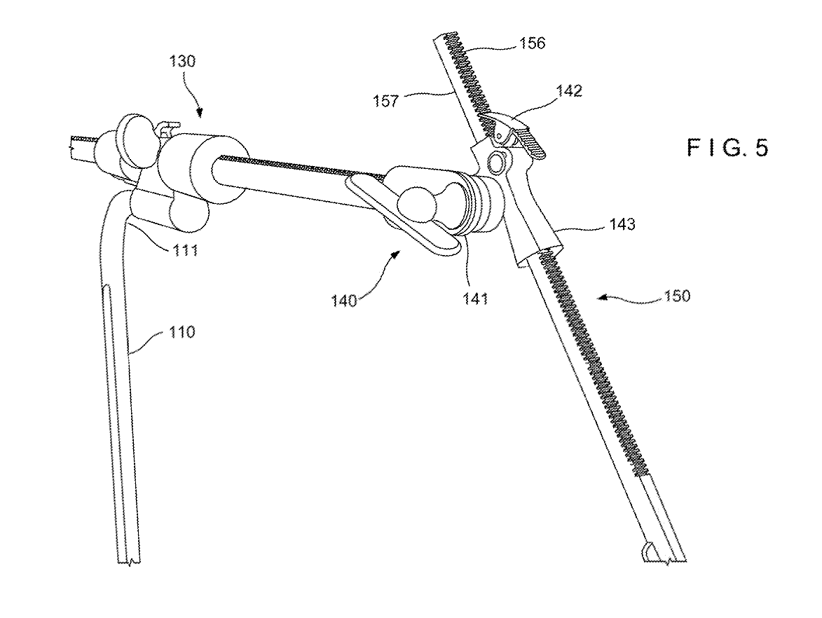

FIG. 5 depicts a close-up view of the secondary adjustment member 140 and the mounting portion 157 of the retractor 150. The flared insertion end 143 of the secondary adjustment member 140 is more clearly visible, the retractor, and the teeth 156 thereof, inserted therethrough. The teeth contacting end of the ratchet 142 (e.g., a pawl) is shown contacting the teeth 156, thereby allowing the retractor to be adjusted upwardly, but not downwardly. To adjust the retractor 150 downwardly, the depression end of the ratchet 142 would be pressed so as to remove contact between the ratchet and the retractor.

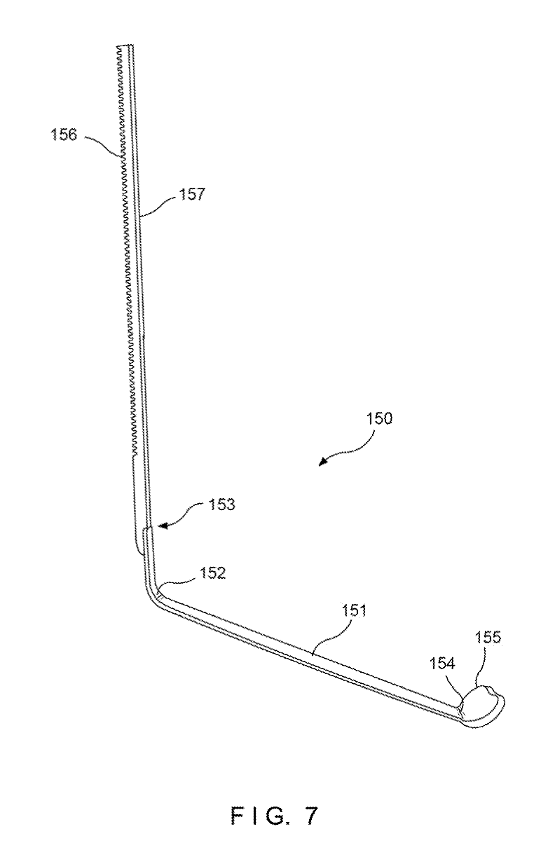

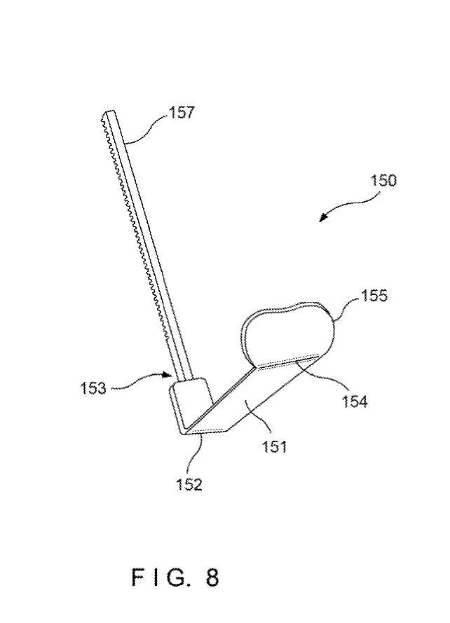

The surgical retractor 150 may include an mounting portion 157, as discussed above, which can be welded (at 153) or other wise affixed or secured to a retraction portion, such as a retractor blase 151, which preferably has a flat and straight base. In one embodiment, the angle between the mounting portion 157 and the blade 151, about bend 152, is approximately 110°. Other angles are possible, as will be discussed in greater detail below, including 90°, or between about 70° or 80° and 130° or 140°, or a range between 70° and 130°. A lip 155 is positioned on the end of the retraction portion 151 opposite the bend 152. A lip 155 is preferably provided at the distal tip of the blade 151 that is inserted within the patient and serves to help retract and retain retracted the desired anatomy for surgery. In some embodiments, the lip 155 may be generally heart-shaped, and bent inwardly at an angle (at 154) to the retraction portion 151, or can have other shape. The preferred lip is preferably bent as an angle upwards from the blade base, towards the side of the mounting portion 157, thereby allowing the horizontal bar 120 to be moved in either direction. Further, FIG. 7 depicts a close-up of a retractor 150 in accordance with the embodiment of FIG. 1. Additionally, FIG. 8 depicts a close-up view, at a different angle, of a retractor 150 in accordance with the embodiment of FIG. 1.

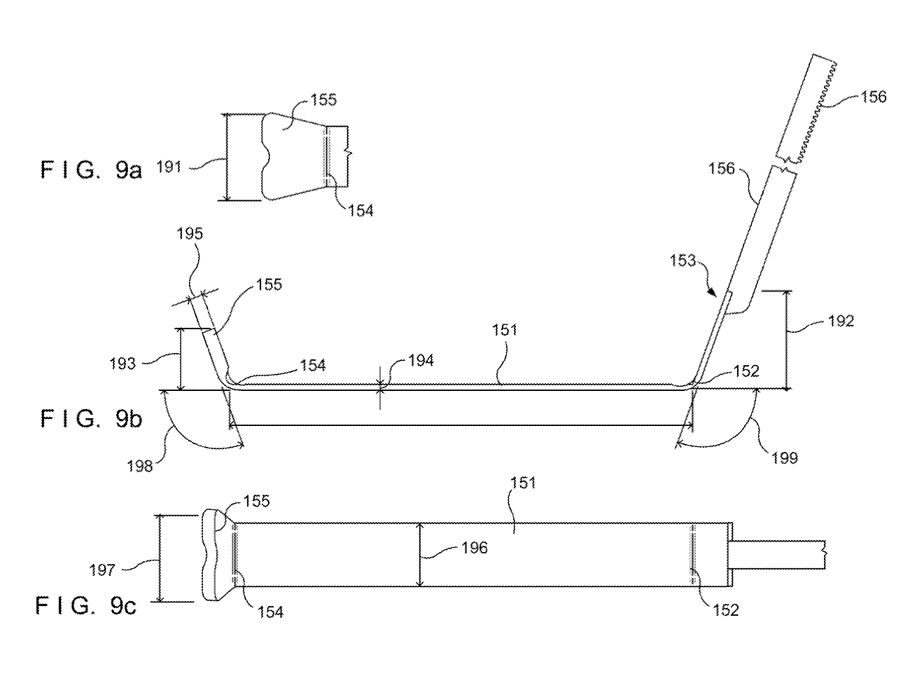

FIG. 9 depicts a schematic illustration of a retractor 150 in accordance with adjustable retractor system of some embodiments of the present disclosure. In particular, lip 155 may be heart-shaped and width 191 preferably of about 30 mm, or about 25 mm-35 mm, or about 20 mm-40 mm. In other embodiments, it may be between 20 and 50 mm in width or other desired sizes. The distance 192 between weld 153 and bend 152 is preferably about 30 mm in one embodiment. In other embodiments, it may be between 2 and 100 mm, for example, although other distances can be used. Several retractors can be provided in a kit, having lengths of the retraction portion 151 such as 150, 170, or 200 mm. In some embodiments, the length may be between 100 and 250 mm, for example. The angle 198 of bend 154 may preferably be 110°, or it may be between 100° and 120°, or 80° and 140°. Other angles can be selected. The bend 152 may be formed of a very small, sharp radius, e.g. 3, 4, 5, 6, or 7 mm, and the angle 199 thereof may be between about 80° and about 140°, or preferably between about 100° and 120°. The portions of the retractor which connect to and terminate at the bend 152, the blade 151 and the mounting portion 157, are substantially straight. The height 193 of the lip 155 may be about 20 mm in a preferred embodiment although other heights, such as between 10 and 30 mm can be used in other embodiments. The thickness of blade 151 and lip 155 (thickness 194, 195, respectively) may be 3 or 4 mm in preferred embodiments, and is typically between about 1 mm and 5 mm in other embodiments. The thickness 194 of the blade 151 can be the same or different than the thickness 195 of the lip 155 or other parts of the retractor. The width 196 of the retraction portion 151 maybe 20 mm in a preferred embodiment. In alternative embodiments it can be between about 10 mm and 30 mm, for example, although other widths can be selected. The width 197 of the lip may be slightly larger than that of the retraction portion at its widest part, for example, preferably 25 mm, or more generally between about 10 mm and 50 mm.

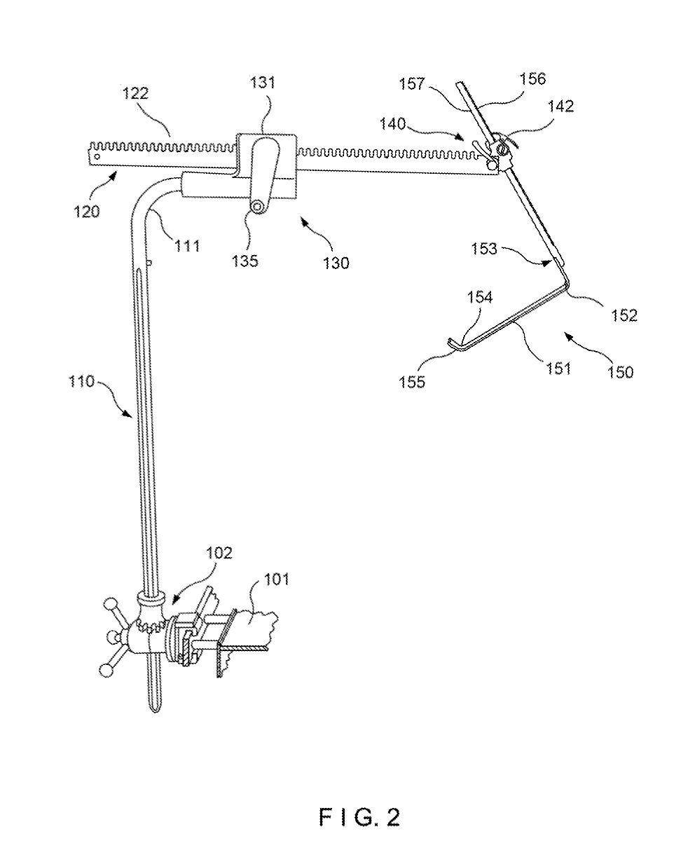

Depicted in FIG. 2 is another example embodiment of an adjustable surgical retractor. In this embodiment, the horizontal bar 120 is configured with notches 122 as opposed to the teeth 121 depicted in FIG. 1. These notches 122 cooperative with an adjustment handle 135 of the horizontal support component 130, so as rotation of the handle causes horizontal movements of the horizontal bar 120 in either direction, depending on which direction the handle 135 is rotated. An internal component of the handle 135 contacts the notches 122 and causes them to be pushed horizontally in either direction when the exterior portion of the handle 135 is rotated. This embodiment provides an alternative rack and pinion arrangement, in which the pinion has over-center positions within the rack so that not ratchet is needed to resist or prevent movement of the rack in either direction without rotating the handle. Further, it is noted that in this depicted embodiment, the bend 152 of the retractor 150 is approximately 90°, as opposed to the approximately 110° of FIG. 1.

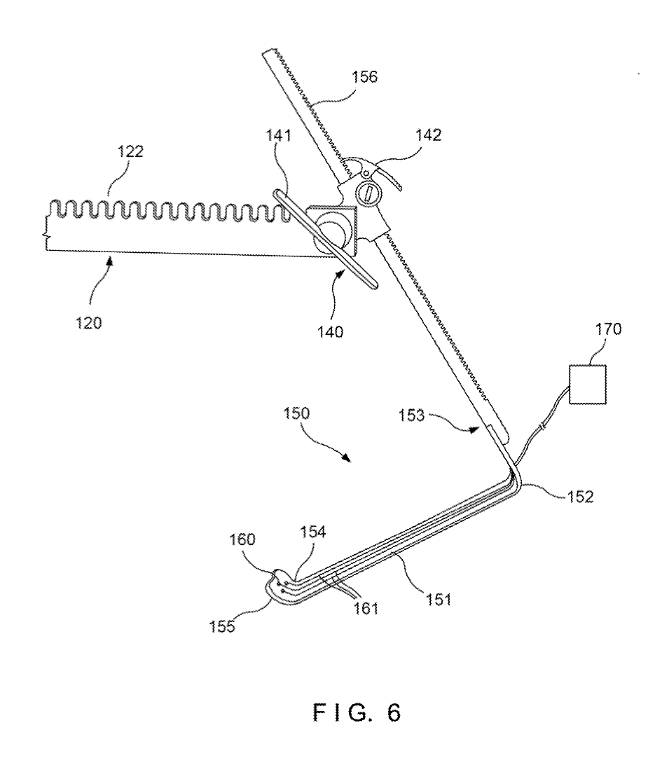

FIG. 6 depicts a close-up view of a retractor 150 in accordance with the embodiment of FIG. 2. On the upper surface of the retraction portion 151 of the retractor 150 are three wires, tubes, or other connections 161 associated with three surgical instruments or sensors, such as suction, a light or other fiber optics, and a sensor such as to interact with a cell of the thyroid or another cell to detect cancer or another condition. The instruments and/or sensors and/or their connections 161 may be inserted through holes 160 of the lip 155 of the retractor 150 or another part of the blade. The cables are connected to instrument bases 170, at another location within the surgical environment.

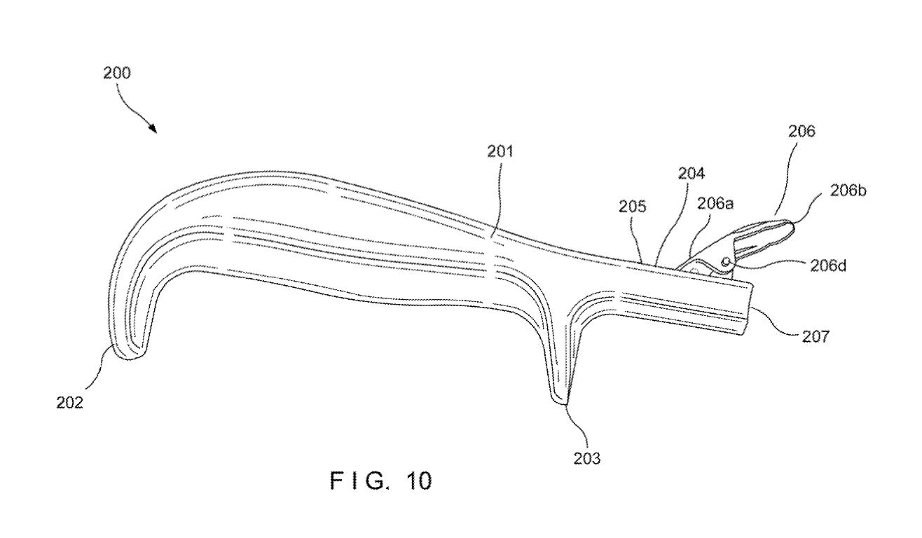

Referring now generally to FIGS. 10-13, a retractor handle 200 suitable for use with the presently disclosed surgical retractor system is depicted. The handle 200 may generally include an axial portion 201 being formed of a generally cylindrically-shaped piece of stainless steel, having irregular diameter across its length so as to conform to the grip of a human hand. On both the proximal and distal ends of the axial portion are located material extensions 202, 203. Lateral extensions 202, 203 extend laterally from the axial portion. Extensions 202, 2003 are spaced apart from one another between about 3 inches-6 inches, or preferably 4 inches-5 inches, to conform to the width of a human hand while the hand is gripping the axial portion 201. Extensions 202, 203 may generally extend between about 0.5 inches-3 inches, or more preferably between about 1 inch-2 inches. A distal extension 204 extends distally from the distal end of the axial portion 201 and generally forms a hollow square of proportions to meet with the mounting portion 157 of a surgical retractor 150. The distal extension 204 generally extends from the axial portion 201 between about 1 inch-4 inches, or preferably between about 1.5 inches-3 inches.

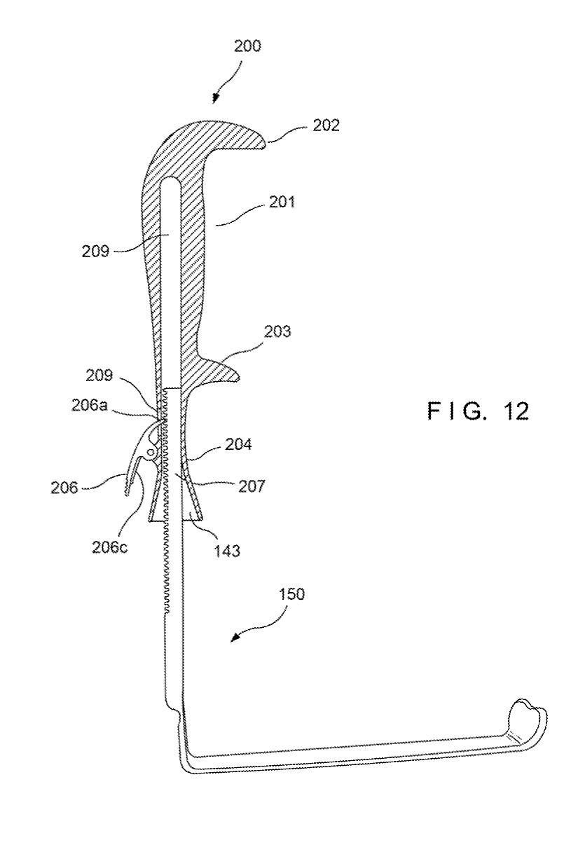

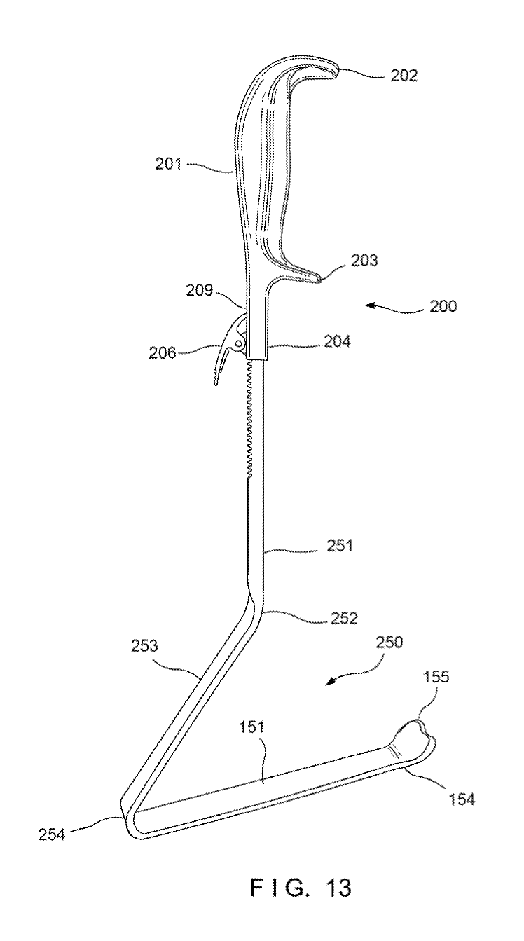

Mounted on an outward surface 205 of the distal extension is a ratchet 206. The ratchet 206 is configured and operates in a manner similar to ratchet 142 of the secondary adjustment member, as described above, having a biasing spring 206c and a pivot point 206d. The ratchet 206, via a teeth contacting end 206a thereof (which may be in the form of a pawl, as discussed above), cooperates with the teeth 156 of the mounting portion 157 of a retractor 150 through an opening 209 on the outward surface 205 of the distal extension 204 (see FIGS. 12 and 13), such that, when the mounting portion is inserted into an open end 207 of the distal extension 204, the retractor 150 by its mounting portion 157 is locked in cooperative association with the handle 200. To release the retractor 150 from the handle 200, a finger pad 206b of the retractor 206 is depressed, thereby releasing contact between the contacting end 206 and the teeth 156, allow the mounting portion 157 to slide-out from within the distal extension 204.

In use, a surgeon or other operator may generally insert the mounting portion 157 of the retractor 150 within the distal extension 204 of the handle 200 to cooperatively lock the two components together. (See FIGS. 12 and 13). The surgeon then grips the axial portion 201 of the handle 200 and manipulates the retractor 150 into position within the patient, i.e. retracting a particular anatomy. Once in the desire position, the handle 200 is released from the retractor by depressing the finger pad 206b, releasing the cooperative association between the mounting portion 157 (and the teeth 156 thereof) and the contacting end 206a of the ratchet 206, allowing the mounting end 157 to slide-out of the distal extension 204 of the handle 200.

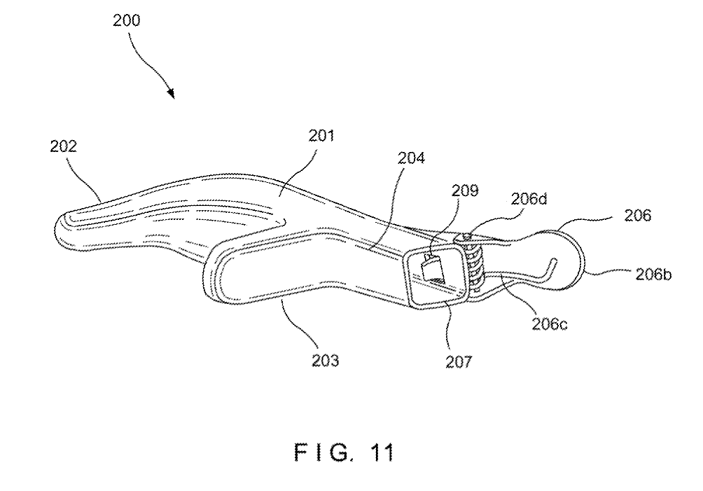

FIG. 11 depicts an axial view of the handle 200, looking into the open end 207 of the distal extension 204. The spring 206c is positioned on the underside of the ratchet 206 and is configured to bias the ratchet about its pivot point 206d in a position to contact teeth 156 when the mounting portion 157 is present.

FIG. 12 depicts a cross-section view of a handle 200. As shown, the inside channel 209 through which the mounting portion of a retractor 150 may be inserted extends substantially the entire length of the axial portion 201. In this embodiment, the handle 200 further includes a flare 143 at distal extension 204. The flare 143 allows easier insertion of the mounting portion of the retractor 150 into the handle 200. As also shown in FIG. 12, the pawl of the ratchet 206 is contacting teeth of the mounting portion, thereby securely holding it in place at the desired length.

FIG. 14 shows an additional embodiment of a retractor 250 connected to a handle 200. The retractor 250 had a configuration especially suited for use with robotically-performed surgery, wherein the retractor 250 is configured to avoid contact with robotic arms during such surgery. The retractor 250 includes a connection portion 251, which may be, for example, welded to, and extending from a mounting portion (as described above, e.g., mounting portion 157). A first bend 252 is angled below the connection portion at a generally obtuse angle, for example, about 170, 160, 150, 140, 130, 120, 110, or 100 degrees from the mounting portion. The first bend 252 is positioned between the connection portion 251 and a side 253, which extend from the bend 252 is positioned between the connection portion 251 and a side 253, which extend from the bend 252 at the desired angle. Side 253 is connected to blade 151 with a second bend 254. The second bend 254 may generally form an acute angle, for example, about 20, 30, 40, 50, 60, 70, or 80 degrees. As described above, in a preferred embodiment, blade 151 may have a heart-shaped lip 155. Alternatively, lip 155 may be a generally rounded shape. The first and second bends 252, 254 allow the side 253 to remain clear of a robotic arm during surgery, while still allowing the blade 151 to be properly positioned. The retractors described in the present disclosure are sufficiently strong to retract a shoulder for providing space for surgical entry through the axilla.

The surgical support, retractors, and handle of the present disclosure may be used in performing various surgeries, including thyroid surgery wherein the thyroid is accessed through the axilla, as well as vaginal and rectal surgeries, and as examples other surgical procedures in which a surgical tool is supported or for example a deep incision or body opening is to be retracted. In one embodiment, a surgeon or surgical technician may position the retractor 150 in the desired location and orientation, sometimes cutting against the bottom side of the retractor blade 151 as the blade 151 is inserted into the incision. The height and angle at which the upright surgical tool support 110 is connected to the rail 101 of the operating table 100 is then adjusted, including by positioning and manipulating the swinger clamp 102 and tightening it onto the rail 101 and upright support 110. Then, the horizontal bar 120 is adjusted horizontally within the horizontal support component 130 and over the patient, in the manner described above rotating the manipuable adjustment component 132, or by simply pulling on the bar 120, which may or may not require depressing the ratchet 133 (depending on which direction the bar is moved, as discussed above). The flared insertion end 143 of the vertical support component may then be guided over the adjustment portion 157 of the retractor 150, an secured in position at the appropriate point along the adjustment portion 157. If the retractor needs to be repositioned once guided within the vertical support component 140, the adjustment portion 141 and/or the ratchet 142 may be manipulated as necessary, in the same manner as the like components of the horizontal support component 130.

In a preferred embodiment of a surgical procedure using the inventive system, thyroid surgery is conducted, in which access to the thyroid is gained through the axilla. This procedure can be performed robotically or manually by a surgeon. The retractor used and the support can be configured to keep the surgical tool support as free from the tissue about the incision, such as an axillic incision, as possible to allow for other surgical equipment, which in some cases may be robotically operated, to more easily access the incision and thyroid or other tissue that needs to be accessed. As shown in FIG. 1, th horizontal bar 120 and surgical retractor is cantilevered from the surgical tool support 110 so that the surgical retractor is entirely supported from the opposite side of the surgical table 100 from the surgical retractor 150. Preferably, the surgical support system is easily adjustable to adapt it's position to a retractor that has already been positioned in an incision in a patient.

The term “substantially,” as used herein to refer to a shape, e.g., substantially semi-cylindrical or semi-circular cross-section, is intended to include variations from the true shape that do not affect the overall function of the device.

The term “about,” as used herein, should generally be understood to refer to both numbers in a range of numerals. Moreover, all numerical ranges herein should be understood to include each whole integer within the range.

While illustrative embodiments of the invention are disclosed herein, it will be appreciated that numerous modifications and other embodiments can be devised by those of ordinary skill in th art. Features of the embodiments described herein can be combined, separated, interchanged, and/or rearranged to generate other embodiments. Therefore, it will be understood that the appended claims are intended to cover all such modifications and embodiments that come within the spirit and scope of the present invention.

1-26. (canceled)

27. A surgical support system, comprising:
a base member having an attachment to mount to a distal side of a surgical table;
an upright support member mountable extending generally upright from the base member;
a horizontal support member mountable extending generally horizontally from the upright support member at a sufficient vertical height above the base member to vertically clear a patient lying on the table and having a sufficient horizontal length to cross over the patient to a proximal side of the surgical table;
a connecting member on a proximal side of the horizontal support member connects a surgical retractor to the horizontal support member such that the surgical retractor disposed on the proximal side of the surgical table is supported entirely from the base member on the distal side of the surgical table via the upright and horizontal support members;
a vertical adjustment member configured for vertically adjusting the retractor with respect to the base and selectively preventing lowering of the blade towards the base; and
a horizontal adjustment member configured for horizontally adjusting the retractor with respect to the base.

28. The surgical support system of claim 27, wherein:
the horizontal adjustment member includes a primary telescoping support component; the horizontal support member is telescopically mounted to the primary telescoping support component; and
the vertical adjustment member includes a secondary telescoping support component mounted to the horizontal support member and being configured for telescopically mounting the surgical retractor thereto, such that telescoping of the horizontal support member with respect to the primary telescoping support component repositions the secondary telescoping support component.

29. The surgical support system of claim 28, wherein the secondary telescoping support component comprises:
a first mounting portion;
a second mounting portion configured for telescopically mounting the surgical tool; and
an articulable joint disposed between and associating the first mounting portion and the second mounting portion for adjusting the angle between the first and second telescoping axes.

30. The surgical support system of claim 29, wherein the articulable joint is releasably lockable for locking the angle.

31. The surgical support system of claim 28, wherein each of the primary and secondary support components comprises an engagement member configured for selectively preventing telescopic extension of the second telescoping support component and surgical retractor, respectively.

32. The surgical support system of claim 31, wherein each engagement member has a configuration in which it allows telescopic refraction of the second telescoping support component and surgical retractor, respectively, while preventing the telescopic extension thereof.

33. The surgical support system of claim 28, wherein the secondary telescoping support component comprises an axially flared end for facilitating axial reception of the surgical retractor into a telescopic mounting bore within the secondary telescoping support component.

34. The surgical support system of claim 33, wherein the axially flared end has a cross-section that flares by about 2 to 10 times in cross-section.

35. The surgical support system of claim 28, wherein:
the horizontal support member is telescopically mounted to the horizontal adjustment member along a generally horizontal telescoping axis;
the horizontal adjustment member is configured to allow the horizontal support member to telescope with respect thereto along the horizontal axis, with said horizontal axis fixed with respect to the base member.

36. The surgical support system of claim 28, wherein:
the handling portion comprises a retractor rack that includes teeth;
the vertical adjustment member includes a ratchet member engageable with the teeth to selectively prevent telescopic downwards extension of the retractor rack while allowing upwards retraction thereof.

37. The surgical support system of claim 28, wherein:
the horizontal support member comprises a refractor rack that includes teeth;
the horizontal adjustment component includes a ratchet member engageable with the teeth to selectively prevent proximal telescopic extension of the retractor rack while allowing upwards retraction thereof.

38. The surgical support system of claim 36, further comprising a handle for grasping by hand to entirely support the surgical refractor and configured for telescopically mounting the surgical retractor thereto and including an engagement member for selectively preventing telescopic extension of the surgical tool rack.

39. The surgical support system of claim 36, wherein the horizontal support member comprises a rack including teeth, and the primary telescoping support component includes an engagement member, which comprises a pinion for telescopically drawing or extending the rack.

40. The surgical support system of claim 36, further comprising retractor holder, wherein the retractor holder includes:
a handle shaft configured for grasping with a hand, the handle shaft defining an internal telescopic bore configured for receiving and guiding a surgical retractor along a telescopic axis; and
a ratchet member associated with the handle shaft for ratcheting engagement with teeth of a handling portion of the surgical retractor to selectively prevent telescopic extension thereof while allowing telescopic retraction.

41. The surgical support system of claim 27, wherein:
the upright support member has a vertical length of at least about 60 cm; and the
horizontal length of the horizontal support member is at least about 40 cm.

42. The surgical support system of claim 27, wherein the base member includes a rail clamp that clamps to a surgical rail of the surgical table.

43. The surgical support system of claim 27, wherein the horizontal support member and surgical refractor mounted thereto are cantilevered from the upright support member to define a free space below the blade for providing clear surgical access.

44. The surgical support system of claim 34, wherein the flared inlet guide and handling portion have different cross-sectional shapes

45. The surgical support system of claim 49, wherein:
the cross-sectional shape of the flared inlet guide is substantially circular; and
the cross-sectional shape of the handling portion is substantially square.

46. The surgical support system of claim 27, further comprising the surgical retractor, which includes:
a handling portion; and
a refractor blade, which blade includes a handling end and a distal end, the handling portion being mountable to the horizontal support member with:
the blade disposed below the horizontal support member,
the handling portion disposed at horizontal distance from the rail clamp greater than a width of the patient, and
the blade extending generally distally and horizontally toward the upright support member so that the distal end is disposed at a horizontal distance from the rail clamp to be positioned within an incision of the patient.

47. The surgical support system of claim 46, wherein the incision is the axilla of the patient.

48. The surgical support system of claim 46, wherein:
the surgical retractor comprises:
a surgical instrument disposed on the blade configured for performing an operation; and
an instrument connector extending along the abutting surface to the surgical instruments.

49. The surgical support system of claim 46, wherein:
the handling portion extends in a first direction for supporting and pulling the retractor blade; and
the surgical retractor further comprises an elbow having a radius of less than about 7 millimeters that connects the handling portion to the blade at an angle to each other.

50. A method of conducting surgery, comprising:
retracting tissue with the retractor of the surgical support system of claim 27; and
conducting robotic surgery while the tissue is retracted.
